(19) United States
(12) Patent Application Publication
(10) Pub. No.: US 2024/0242788 A1
(43) Pub. Date: Jul. 18, 2024

(54) Method and System for Recognizing Chemical Information from Document Images

(71) Applicant: LIMITED LIABILITY COMPANY SYNTELLY, Moscow (RU)

(72) Inventors: Ivan Sergeevich KHOKHLOV, Savinskij (RU); Lev Valer'evich KRASNOV, Novosibirsk (RU); Maxim Velerievich FEDOROV, Novoivanovskoye (RU); Sergey Borisovich SOSNIN, Odincovo (RU)

(21) Appl. No.: 18/574,499

(22) PCT Filed: Jul. 8, 2021
(86) PCT No.: PCT/RU2021/000294

§ 371 Date: Dec. 27, 2023

(30) Foreign Application Priority Data
Jun. 28, 2021 (RU) 2021118778

(51) Int. Cl. G16C 20/70; G06V 30/413

(52) U.S. Cl. CPC G16C 20/70; G06V 30/413

(57) ABSTRACT

The present disclosure is related to the field of data recognition. The computer-implementable method includes the following steps: inputting an image of a document page to a detector; the detector identifies fragments on the page; obtaining coordinates of the fragment on the page for each identified fragment; and classifying the fragments; the structure recognition unit recognizes the chemical structure for each fragment; inputting identified fragments of the reaction arrows to an arrow recognition unit; obtaining coordinates on the page for each arrow and reaction attributes; transmitting to an input of a reaction recognition unit the coordinates on the page for each fragment of the recognized chemical structures; and based on the obtained data the reaction recognition unit determines how the arrows relate to the recognized chemical structures; as a result, based on the recognized data for the image of the document page, obtaining recognized chemical structures.

### FIELD OF THE INVENTION

The present disclosure is related to the field of data recognition, in particular to a method of recognizing chemical information from document images and a system for implementing the same method.

The present solution can be used at least in pharmaceutical companies to collect data on various chemical information, for example, chemical compounds presented in different formats, chemical reactions and additional chemical information, in other fields of technology in which it is necessary to collect data on such chemical information. The present solution can also be used by chemical information vendors to fill databases.

### BACKGROUND OF THE INVENTION

The prior art discloses a solution CN111860507A, publication date 30 Oct. 2020, describing a method for extracting the molecular structural formula of a compound image based on adversarial learning, which belongs to the field of deep learning, image recognition and compound molecular formula extraction. The method for extracting the molecular structural formula of a compound image based on adversarial learning comprises the following steps: constructing a dataset of data pairs consisting of compound images and SMILES codes; establishing a countermeasure network consisting of a SMILES code generator and a SMILES code recognizer, and adversarial learning of the proposed neural network model.

The prior art, international application WO2019148852A1, publication date 8 Aug. 2019, discloses a method of recognizing chemical information from images of hand-drawn images by identifying structures, identifying handwritten script, identifying atoms corresponding to structures, identifying bonds using deep learning techniques.

The prior art, patent WO2019148852A1, publication date 8 Apr. 2020, discloses a device for electronically identifying and composing chemical structures detected in a repository in which electronic files are stored. The optical structure recognition module identifies a plurality of possible chemical structures in the electronic files of the repository, wherein at least one of the electronic files contains non-embedded images of the chemical structures identified by the optical structure. The recognition module outputs (for each identified potential chemical structure) a chemical structure object with a related set of properties, including the number of carbon atoms (e.g., number of heteroatoms, number of bonds, number of bonds of the selected bond order, number of rings and formula weight). The optical structure recognition module also applies (for each derived chemical structure object) one or more filters, including a filter to exclude objects identified as having less than a selected number of carbon atoms, wherein the selected number of carbon atoms is configured and set by the user based on the expected contents of electronic files, and stores objects not removed by one or more filters in a searchable electronic database of identified objects.

The prior art discloses a solution CN112818645A, publication date 18 May 2021, describing a chemical information extraction method and a chemical information extraction device, wherein obtaining a chemical engineering document, identifying an image and a text from the chemical engineering document, extracting a chemical structure and a label for the chemical structure, establishing a mapping relation between the chemical structure and the label, extracting a chemical object and relations between the chemical objects from the text.

In [1] the authors presented a new model of DECIMER (Deep IEarning for Chemical ImagE Recognition). A network based on the Transformer neural architecture is disclosed. The Transformer neural network can recognize molecular structures as SMILES notation with more than 96% accuracy for images of chemical structures without stereochemical information and with more than 89% accuracy for images with stereochemical information.

In [2], the authors address the problem of image-to-text translation specifically for molecular structures, where the result will be a predicted chemical designation in InChI format for a given molecular structure. Current approaches are mostly rule-based or methodology based on CNN+RNN. However, according to the authors of [2], they show worse results on noisy images and images with few distinguishable details. To overcome these limitations, the authors proposed an end-to-end transformer model. Compared to the attention-based methods, the proposed model shows better performance.

In [3], the authors present a fast and accurate model combining a deep convolutional neural network that learns from molecular images and a pre-trained decoder that translates the hidden representation into a SMILES molecular representation. The method, named Img2Mol by the authors, is capable of correctly recognizing up to 88% of molecular images and converting to SMILES.

In chemical journals, scientific articles, patents, technical reports, dissertations and other chemical documents, key information is presented in the form of images not only of molecular structures in a standardized format, but also of structures written in a non-standardized format, indicating “pseudochemical groups” R1, R2, etc. In general terms, such structures are called Markush structures. Besides, chemical formulas often include designations of chemical groups in the form of abbreviations—for example, “Ph-”, “MeO—”. In addition, key chemical information is also presented in the form of chemical reactions. However, solutions in the prior art lack the ability to optically recognize chemical information in the form of structures written in a non-standardized or abbreviated format, as well as in the form of chemical reactions, from the original optical scans of various chemical documents.

Optical recognition of this kind of information is a difficult task. Technical problem, for solving of which the claimed technical solution is intended, is the recognition of chemical information containing both chemical structures written in standardized and non-standardized or abbreviated format, as well as chemical reactions from chemical documents.

### SUMMARY OF THE INVENTION

The technical result of the claimed invention consists in providing automatic chemical information recognition from document images containing both chemical structures recorded in standardized and non-standardized or abbreviated format, as well as chemical reactions, reducing time and increasing accuracy of the chemical information recognition from the document images. The additional technical result consists in increasing the performance of the computing system while solving the present problem, i.e., the solution allows to process documents in a shorter amount of time to achieve a recognition result, thereby reducing the load on the central processing unit of the computing device.

The claimed result is achieved due to implementation of the computer-implementable method for recognizing chemical information from document images, wherein a computing device including a processor and a memory stores in the memory computer-executable instructions, and perform the instructions including the following steps:
inputting an image of a document page to a detector; the detector identifies one or more fragments on the page using the first neural network, wherein the fragments contain chemical information; obtaining coordinates of the fragment on the page for each identified fragment; and classifying the fragments into at least the following categories: chemical structure, reaction arrow;
inputting one or more identified fragments of the chemical structures to a structure recognition unit, wherein each fragment is an image; the structure recognition unit recognizes the chemical structure for each fragment using the second neural network;
inputting one or more identified fragments of the reaction arrows to an arrow recognition unit; the arrow recognition unit determines the arrow type using the third neural network; and obtaining coordinates on the page for each arrow and reaction attributes using the fourth neural network;
transmitting to an input of a reaction recognition unit the coordinates on the page for each fragment of the recognized chemical structures, the appropriate recognized chemical structures, the coordinates on the page for each reaction arrow, the arrow type, the reaction attributes; and based on the obtained data the reaction recognition unit determines how the arrows relate to the recognized chemical structures;
as a result, based on the recognized data for the image of the document page, obtaining one or more recognized chemical structures, the coordinates on the page for each recognized chemical structure, recognized relationships between agents involved in the chemical reaction, represented as the chemical structures, the coordinates on the page for each recognized relationship.

In another example embodiment, the chemical structure is at least a chemical compound, Markush structure, chemical structure with substituents.

In another example embodiment, further identifying fragments containing an additional information facilitating the recognition of the reactions.

In another example embodiment, the additional information includes at least the following: title, legend.

In another example embodiment, further the detector for each identified fragment determines confidence—a number from 0 to 1, which evaluates the validity of the identified fragment, where 0—absolutely not confident, 1—completely confident.

In another example embodiment, filtering the identified fragments according to a preset confidence threshold.

In another example embodiment, setting the confidence threshold for each fragment category.

In another example embodiment, the first neural network is a Faster R-CNN neural network or another convolutional network of equal or greater power.

In another example embodiment, the second neural network is a neural network based on a transformer architecture, and the structure recognition unit comprises a convolutional unit and the transformer decoder.

In another example embodiment, the convolutional unit is a ResNet-50 network without the last two layers or another convolutional network processing images.

In another example embodiment, the recognized chemical structure is a text sequence that uniquely describes the chemical structure.

In another example embodiment, describing the chemical structure as a text sequence by a SMILES modification, wherein the SMILES modification is able to describe Markush structures and chemical structures with substituents.

In another example embodiment, implementing a mechanism for converting the SMILES modification capable of describing Markush structures and chemical structures with substituents into SMILES and for back converting.

In another example embodiment, the third and fourth neural networks are convolutional neural networks based on ResNet.

In another example embodiment, classifying the arrows according to the following types: straight arrow, not a straight arrow.

In another example embodiment, the agents involved in the chemical reaction are initial agents of the chemical reaction, products of the chemical reaction.

The claimed result is also achieved due to the system for recognizing chemical information from document images, comprising:
a detector;
a structure recognition unit;
an arrow recognition unit;
a reaction recognition unit;

and wherein a computing device including a processor and a memory stores in the memory computer-executable instructions, and the computing device performs the method according to the method above.

### DESCRIPTION OF THE DRAWINGS

Implementation of the invention will be further described in accordance with the attached drawings, which are presented to clarify the invention chief matter and by no means limit the field of the invention.

The drawing FIGS. 1-4 depict implementations in accordance with the present invention:

FIG. 1 illustrates a block diagram of the system for recognizing chemical information from document images.

FIG. 2 illustrates a block diagram of the modified transformer.

FIG. 3a, 3b, 3c, 3d illustrate an implementation example of the system for recognizing chemical information from document images.

FIG. 4 illustrates a general block diagram of the computing device for implementing the present invention.

### DETAILED DESCRIPTION OF THE INVENTION

Numerous implementation details intended to ensure clear understanding of this invention are listed in the detailed description of the invention implementation given next. However, it is obvious to a person skilled in the art how to use this invention as with the given implementation details as without them. In other cases, the well-known methods, procedures and components have not been described in detail so as not to obscure unnecessarily the present invention.

Besides, it will be clear from the given explanation that the invention is not limited to the given implementation. Numerous possible modifications, changes, variations and replacements retaining the chief matter and form of this invention will be obvious to persons skilled in the art.

The present invention is an automated mechanism for recognizing chemical information from actual chemical documents.

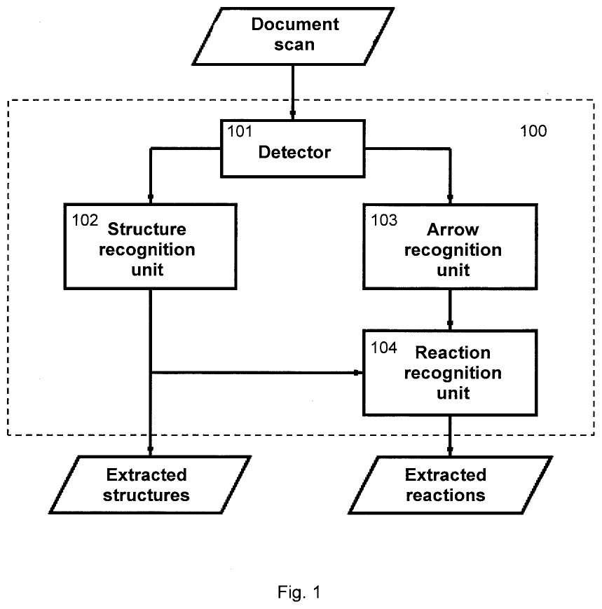

FIG. 1 illustrates the architecture of the chemical information recognition system from document images. The system (100) comprises the following main units:
Detector (101) that locates individual elements of chemical information on a scan of a chemical document using the neural network based on the “Faster R-CNN” architecture or any other more powerful convolutional network.
Structure recognition unit (102), which, using the neural network based on the modified Transformer architecture, solves the problem of translating an image into text (image captioning). The Transformer architecture is modified to provide an image rather than a sequence to the network input. There is no encoder block in the modified Transformer architecture.
Arrow recognition unit (103) which classifies arrows depicting the direction of reaction using the neural network. The neural network recognizes the coordinates of the start and the end of the arrows, as well as the attributes of the reactions.
Reaction recognition unit (104) which determines how the arrows bind the recognized chemical structures.

The system input is provided with a scan of the document page, which is transmitted to the detector (101). The detector (101) using the neural network finds rectangular areas on the page containing chemical information—molecules and reaction arrows, as well as additional information that helps in recognizing reactions, for example, titles and scheme legends.

The detector (101) is based on the Faster R-CNN network, but can equally be implemented based on any neural network architecture that solves the detection problem (YOLO, SDD, EfficientDet, etc.).

For each fragment found, the following are returned:
1. Coordinates on the original page;
2. Category (molecule, arrow, title, legend);
3. Confidence—a number from 0 (absolutely not confident) to 1 (completely confident).

The obtained objects are filtered according to the preset confidence threshold (for example, 0.8), i.e. a fragment with a confidence value less than 0.8 will be discarded as unreliable. The threshold can be set differently for each category of fragments.

The data used to train the detector (101) were manually marked up using a markup interface specially written for this purpose. A total of 2,500 pages of articles were marked. Further filling of the training data set is possible in semi-automatic mode, when the detector prediction is corrected manually.

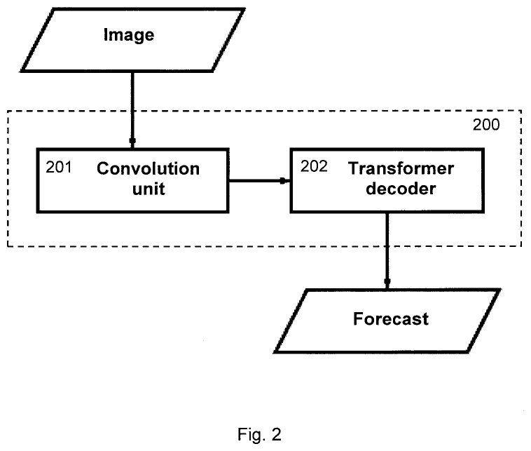

The structure recognition unit (102) converts images of chemical structures in chemical documents into structures as a text sequence using the neural network. The chemical structure images that are fed to the structure recognition unit (102) describe, for example, the spatial structures of molecules, initial agents involved in chemical reactions, products of chemical reactions, etc. In addition, the structure recognition unit (102) recognizes chemical structures that are depicted in both standardized and non-standardized or abbreviated formats. The neural network is implemented based on the Transformer architecture. A conventional Transformer solves the problem of sequence2sequence, i.e., translation from one sequence to another, such as machine translation. In the present invention, an image (2D-sequence) rather than a 1D-sequence is input, so the modified version of the Transformer is used (FIG. 2). The modified transformer (200) contains the convolution unit (201) and the transformer decoder (202). The encoder unit used in the conventional transformer is completely replaced by the convolution unit (201). The ResNet-50 without the last two layers is used as the convolution unit (201). Thus, if an image of 384×384 is fed, the output from the convolution unit (201) is a 512×48×48 matrix, which is equivalent to a transformer encoder with a depth of 512 and an input sequence length of 48. At the same time, convolution unit (201) can be not only ResNet, but any other convolutional network, working with the image, from a set of variants, such as EfficientNet, DenseNet, etc.

The Transformer (200) converts the image into a text sequence unambiguously describing the spatial structure of the molecule. The language to describe the molecule can be any representation: SMILES and its variations (DeepSMILES, SELFIES), as well as InChI or IUPAC name. SMILES-based variants are preferred because they are the most concise and reflect the structure directly.

The FG-SMILES (Functional Group SMILES) language has been developed for textual representation of Markush structures as well as structures with substituents. FG-SMILES is an extension of the usual textual description language for chemical structures SMILES. FG-SMILES allows to write both structures with functional groups in abbreviated form and Markush structures. The CXSMILES language, a well-known analogue for recording substituent groups and Markush structures, is too verbose in comparison with FG-SMILES, and also does not allow to record R-groups in undefined position. In addition, for FG-SMILES a conversion mechanism to and from SMILES is implemented.

This notation allows to write functional groups as substituted atoms, for example:
[Et]N([Et])CCCNc1nc([X])nc([R3])c1[R2]

In classical SMILES, inorganic atoms, ions, isotopes, and stereo atoms are written in square brackets. In this modification, the abbreviated names of the functional groups as well as the R-groups are written in a similar way. A method of linking an R-group to a cycle rather than to a specific position in the cycle is also presented (if there is a need to show that the R-group is in an undefined position in the cycle).

There is also a CXSMILES extension that solves a similar problem (except for an undefined position), but the corresponding representation is not intuitive and many times longer, which negatively affects the ability to use CXSMILES in machine learning. CXSMILES string corresponding to the example above:
*c1nc(*)c(*)c(NCCCN(*)*)n1|atomProp:0.dummyLabel.X:4.dummyLabel.R3:6.dummyLabel.R2:13.dummyLabel.Et:14.dummyLabel. Et|

The implemented SMILES↔FG-SMILES conversion mechanism allows to find known functional groups in SMILES and replace them. The list of functional groups includes more than 100 groups. These are not all possible groups, but this list covers the vast majority of real examples from the articles, and can also be supplemented.

The data for training the structure recognition model is generated artificially using a data generator based on the method of creating an artificial dataset imitating data from real scientific articles. This is due to the fact that the authors of real chemical articles take considerable liberties in depicting structures. In addition to differences in fonts, line thicknesses, and indents, there are often elements of artistic design. To make the model robust to such features of real data, the generator applies random nonlinear geometric distortions to the images, and adds random chemically meaningful debris—fragments of other molecules, arrows, and inscriptions, to the free space of the image.

The data generator produces a random modification of the base molecule, produces an image of the modified molecule, and the corresponding FG-SMILES.

The generator receives a SMILES string as input. Then it looks for functional groups in the corresponding molecule, and randomly chosen part of them is replaced by a short representation, thus forming FG-SMILES. Some of the methyl substituents are replaced by a random R-group. The molecule is then rendered by the RDKit library according to functional group substitutions that have been made. The resulting image-FG-SMILES pairs are used to train the model.

The trained model returns the forecast as an FG-SMILES string, and the confidence in the forecast is a number from 0 to 1. A strong relationship was found between the returned confidence value and the correctness of the answer. Disregarding the confidence values, the base model has an accuracy of about 90% on test data. Accuracy refers to the proportion of complete correspondences between the real value and the prediction, where even the indices and numbers of dashes near the R-groups are differentiated. If examples with a confidence value less than 0.98 are discarded, then 10% of the examples are discarded, but in the remaining examples the accuracy becomes 97%. According to the threshold 0.99, 15% is cut, the accuracy on the remaining ones is 98.6%, according to the threshold 0.995, 22% is cut, the accuracy on the remaining 99.8%.

In fact, this allows to achieve absolute accuracy, if the task is not to recognize everything. In the task of mass recognition of structures and reactions for automatic filling of databases, it does not matter in principle if some structures will be rejected, but it is of fundamental importance to prevent false data from entering the databases. Thus, cutting along the threshold allows achieving almost absolute accuracy.

Fragments of the original page recognized by the detector (101) as “reaction arrows” are transmitted to the arrow recognition unit (103). The first network in the arrow recognition unit determines whether the fragment is a straight arrow indicating the reaction. The architecture of the network is the simplest convolutional network based on ResNet. The network was trained on 10,000 fragments obtained from the detector (101) and marked manually. The network returns “Yes” if the fragment is a single straight arrow signifying an irreversible reaction, and “No” in other cases. Thus, variants are excluded when the arrow represents a reversible reaction, an equilibrium state, a reaction mechanism, or the fragment is erroneously returned by the detector (101).

The coordinates of the start and the end of the arrow are recognized by another convolutional network based on the ResNet with four outputs, indicating the position of the X and Y coordinates of the start and the end of the arrow, respectively, in fractions of the length/width of the fragment. 7,000 real fragments obtained using a detector (101) for which the first network returned “Yes” were manually marked to train the network.

The tasks facing both networks are not complex by the standards of modern technologies, unlike the detector (101) or the structure recognition unit (102), both networks have learned with an accuracy close to 100%.

The reaction arrow recognition unit (103) determines the coordinates of the start and the end of the arrows on the original page. Together with the rectangle coordinates of the recognized structures, this information is transmitted to the reaction recognition unit (104), which is a logical algorithm that determines for each arrow whether there is a recognized structure for it near the start and the end of the arrow. If a plausible option is found, the structure at the start of the arrow is considered a reagent, and the structure beyond the end of the arrow is considered a reaction product. The reaction recognition unit (104) generates a result in the form of ready reactions. The output of unit (104) generates a list of recognized structures with coordinates and confidence values, as well as a list of reactions.

Thus, after processing a page scan, the output of the system (100) provides recognized chemical structures, coordinates on the page for each recognized chemical structure, recognized relationships between reactants, agents and chemical reaction products represented as chemical structures, coordinates on the page for each recognized relationship.

An example of recognition of chemical information from document images, which is presented to explain the matter of the invention and in no way limits the scope of the invention.

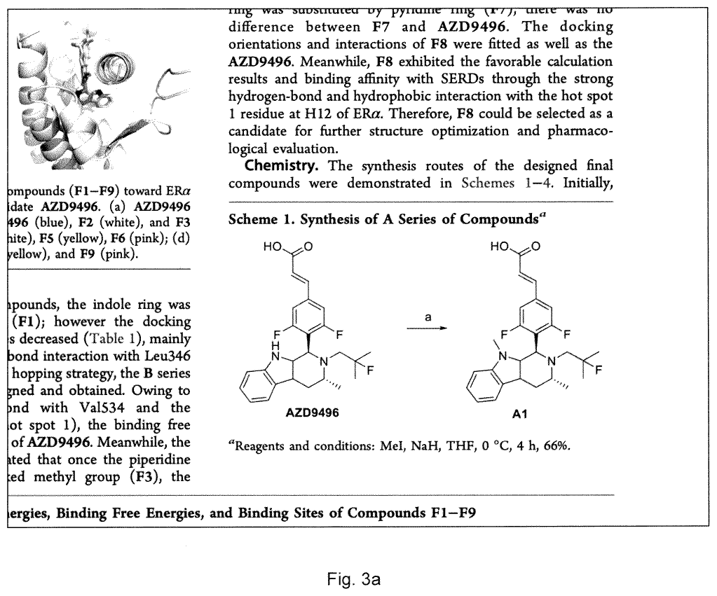

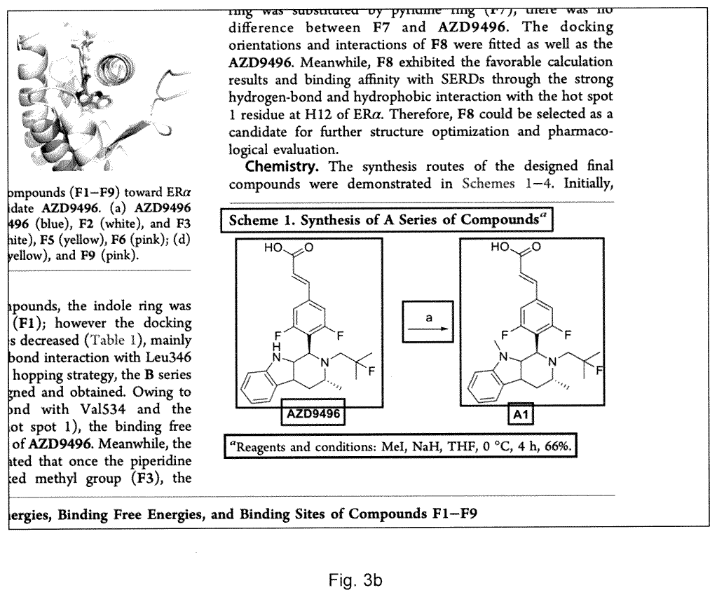

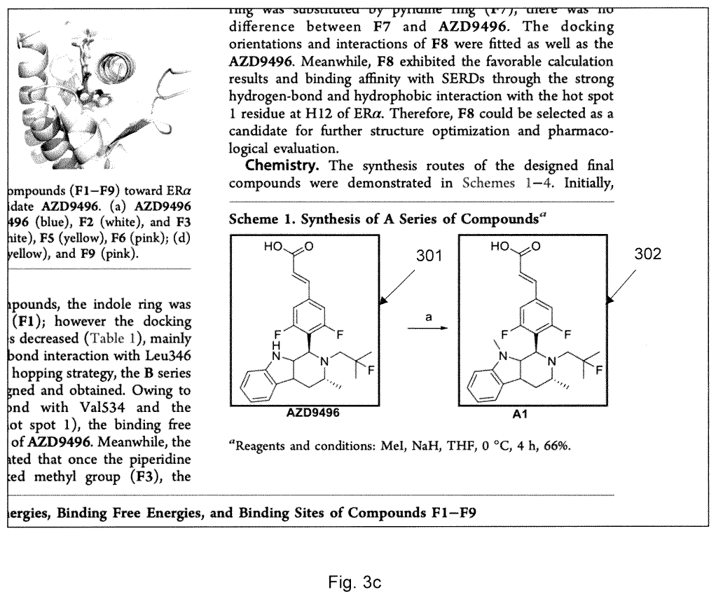

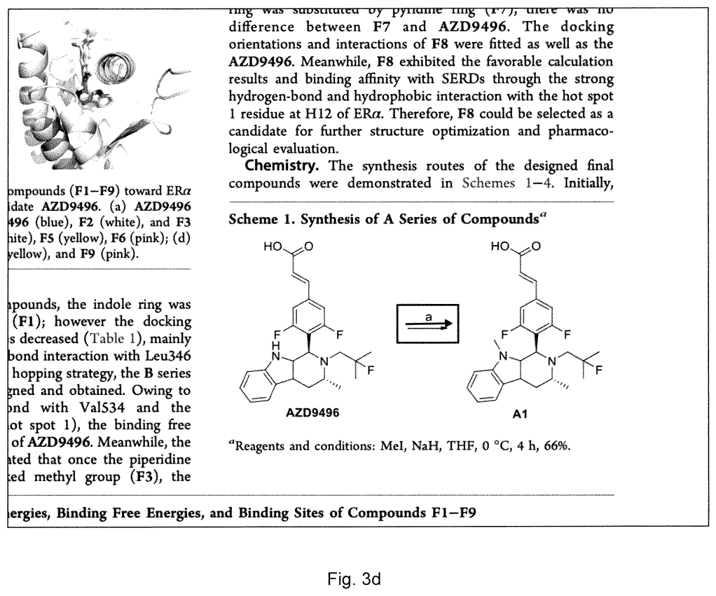

The input of the detector (101) is an image of a page of a document (FIG. 3a) containing chemical information. The detector (101) using the first neural network identifies fragments on the page containing chemical information. In FIG. 3b, the fragments identified by the detector (101) are highlighted by rectangles. For each identified fragment, the coordinates of the fragment on the page are obtained; and the fragments are classified into the following categories: chemical structure—rectangles with molecular structure, reaction arrow—rectangle with arrow, additional information—two small rectangles “AZD9496”, “A1”, and one long rectangle containing additional information about the chemical reaction, and the title rectangle “Scheme 1” (FIG. 3b). The rectangles of the molecules have the image class, the rectangle with the arrow is the condition class, the two small rectangles and one long rectangle are the description class, and the header rectangle is the legend class.

One or more identified chemical structure fragments are input to the structure recognition unit (102), wherein each fragment is an image (FIGS. 3c, 301, 302); the structure recognition unit (102) recognizes the chemical structure for each fragment using a second neural network.

For fragment (301) the unit recognizes FG-SMILES: FC=1C═C(C═C(C1[C@H]1N([C@@H](CC2C1NC1=CC═CC═C21)C)CC(C)(C)F)F)/C=C/C(═O)O

For fragment (302) the unit recognizes FG-SMILES: C[C@@H]1CC2c3ccccc3N(C)C2[C@@H](c2c(F)cc(/C=C/C(═O)O)cc2F)N1CC(C)(C)F

The identified reaction arrow fragment (FIG. 3d) is transmitted to the input of the arrow recognition unit (103). The arrow recognition unit (103) uses the third neural network to determine the type of the arrow—the straight arrow, and uses the fourth neural network to obtain the coordinates of the start and the end of the arrow on the page, and reaction attributes.

The coordinates on the page of each fragment of the recognized chemical structures, the corresponding recognized chemical structures, the coordinates on the page of each reaction arrow, the arrow type, and the reaction attributes are sent to the reaction recognition unit (104) input; and based on the data received, the reaction recognition unit (104) determines how the arrows link the recognized chemical structures.

As a result, based on the recognized data for the image of the document page, obtaining one or more recognized chemical structures, the coordinates on the page for each recognized chemical structure, recognized relationships between agents involved in the chemical reaction, represented as the chemical structures, the coordinates on the page for each recognized relationship.

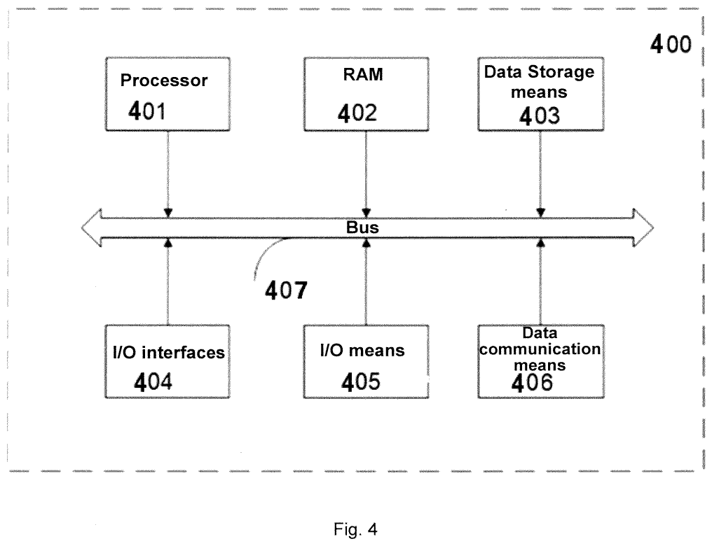

In FIG. 4 is presented a general diagram of the computing device (400) providing data processing necessary to implement the disclosed solution.

In general, the device (400) comprises such components as: one or more processors (401), at least one memory (402), data storage means (403), input/output interfaces (404), input/output means (405), networking means (406).

The device processor (401) executes main computing operations, required for functioning the device (400) or functionality of one or more of its components. The processor (401) runs the required machine-readable commands, contained in the random-access memory (402).

The memory (402), typically, is in the form of RAM and comprises the necessary program logic ensuring the required functional.

The data storage means (403) could be in the form of HDD, SSD, RAID, networked storage, flash-memory, optical drives (CD, DVD, MD, Blue-Ray disks), etc. The means (403) enables to store different information, e.g. the above-mentioned files with user data sets, databases comprising records of time intervals measured for each user, user identifiers, etc.

The interfaces (404) are the standard means for connection and operation with server side, e.g. USB, RS232, RJ45, LPT, COM, HDMI, PS/2, Lightning, FireWire, etc.

Selection of interfaces (404) depends on the specific device (400), which could be a personal computer, mainframe, server cluster, thin client, smartphone, laptop, etc.

Keyboard should be used as data input/output means (405) in any embodiment of the system implementing the described method. There could be any known keyboard hardware: it could be as integral keyboard used in a laptop or netbook, as a separate device connected to a desk computer, server or other computer device. Provided that, the connection could be as hard-wired, when the keyboard connecting cable is connected to PS/2 or USB-port, located on the desk computer system unit, as wireless, when the keyboard exchanges data over the air, e.g. radio channel with a base station, which, in turn, is connected directly to the system unit, e.g. to one of USB-ports. Besides a keyboard the input/output means could also include: joystick, display (touch-screen display), projector, touch pad, mouse, trackball, light pen, loudspeakers, microphone, etc.

The networking means (406) are selected from devices that provide network data reception and transmission, e.g. Ethernet-card, WLAN/Wi-Fi module, Bluetooth module, BLE module, NFC module, IrDa, RFID module, GSM modem, etc. Making use of the means (405) provides an arrangement of data exchange through wire or wireless data communication channel, e.g. WAN, PAN, LAN, Intranet, Internet, WLAN, WMAN or GSM, 3G, 4G, 5G.

The components of the device (400) are interconnected by the common data bus (407).

The present application discloses a preferred disclosure of the implementation of the claimed technical solution, which should not be used as limiting other, particular embodiments thereof, which are within the claimed scope of protection and are obvious to persons skilled in the art.

One skilled in the art should understand that various variations of the method and system do not alter the matter of the invention, but only determine its specific embodiments and implementations.

### REFERENCES CITED

[1] Rajan et al., DECIMER 1.0: Deep Learning for Chemical Image Recognition using Transformers, 2021 https://chemrxiv.org/ndownloader/files/27775521
[2] Sundaramoorthy et al., End-to-End Attention-based Image Captioning», 30.04.2021 https://arxiv.org/pdf/2104.14721.pdf
[3] Clevert et al., Img2Mol-Accurate SMILES Recognition from Molecular Graphical Depictions, 2021 https://chemrxiv.org/ndownloader/files/27273986

1. A computer-implementable method for recognizing chemical information from document images, wherein a computing device including a processor and a memory stores in the memory computer-executable instructions, and perform the instructions comprising the following steps:
inputting an image of a document page to a detector, the detector identifies one or more fragments on the page using the first neural network, wherein the fragments contain chemical information;
obtaining coordinates of the fragment on the page for each identified fragment;
classifying the fragments into at least the following categories: chemical structure and reaction arrow;
inputting one or more identified fragments of the chemical structures to a structure recognition unit, wherein each fragment is an image, wherein the structure recognition unit recognizes the chemical structure for each fragment using the second neural network;
inputting one or more identified fragments of the reaction arrows to an arrow recognition unit, wherein the arrow recognition unit determines the arrow type using the third neural network;
obtaining coordinates on the page for each arrow and reaction attributes using the fourth neural network;
transmitting to an input of a reaction recognition unit the coordinates on the page for each fragment of the recognized chemical structures, the appropriate recognized chemical structures, the coordinates on the page for each reaction arrow, the arrow type, the reaction attributes, and based on the obtained data the reaction recognition unit determines how the arrows relate to the recognized chemical structures; and
based on the recognized data for the image of the document page, obtaining one or more recognized chemical structures, the coordinates on the page for each recognized chemical structure, recognized relationships between agents involved in the chemical reaction, represented as the chemical structures, and the coordinates on the page for each recognized relationship.

2. The method of claim 1, wherein the chemical structure is at least one of a chemical compound, a Markush structure, and a chemical structure with substituents.

3. The method of claim 1, wherein further identifying fragments containing an additional information facilitating the recognition of the reactions.

4. The method of claim 3, wherein the additional information includes at least the following: title, legend.

5. The method of claim 1, wherein the detector for each identified fragment further determines confidence—a number from 0 to 1, which evaluates the validity of the identified fragment, wherein 0 is absolutely not confident, and 1 is completely confident.

6. The method of claim 5, further comprising filtering the identified fragments according to a preset confidence threshold.

7. The method of claim 6, further comprising setting the confidence threshold for each fragment category.

8. The method of claim 1, wherein the first neural network is a Faster R-CNN neural network or another convolutional network of equal or greater power.

9. The method of claim 1, wherein the second neural network is a neural network based on a transformer architecture, and the structure recognition unit comprises a convolutional unit and the transformer decoder.

10. The method of claim 9, wherein the convolutional unit is a ResNet-50 network without the last two layers or another convolutional network processing images.

11. The method of claim 1, wherein the recognized chemical structure is a text sequence that uniquely describes the chemical structure.

12. The method of claim 11, further comprising describing the chemical structure as a text sequence by a SMILES modification, wherein the SMILES modification is able to describe Markush structures and chemical structures with substituents.

13. The method of claim 12, further comprising implementing a mechanism for converting the SMILES modification capable of describing Markush structures and chemical structures with substituents into SMILES and for back converting.

14. The method of claim 1, wherein the third and fourth neural networks are convolutional neural networks based on ResNet.

15. The method of claim 1, wherein the classifying the arrows step includes classifying the arrows according to the following types: straight arrow, and not a straight arrow.

16. The method of claim 1, wherein the agents involved in the chemical reaction are initial agents of the chemical reaction, and products of the chemical reaction.

17. A system for recognizing chemical information from document images, comprising:
a detector;
a structure recognition unit;
an arrow recognition unit;
a reaction recognition unit; and
wherein a computing device including a processor and a memory stores in the memory computer-executable instructions, and the computing device performs the method according to claim 1.
